(19) United States
(12) Patent Application Publication
(10) Pub. No.: US 2002/0122771 A1
(43) Pub. Date: Sep. 5, 2002

(54) Spray hydrogel wound dressings

(76) Inventors: Troy Holland, Suwanee, GA (US); Hassan Chaouk, Atlanta, GA (US); Bruktawit Asfaw, Atlanta, GA (US); Stephen Goodrich, Tucson, AZ (US); Adrian Hunter, Dunwoody, GA (US); Vimala Francis, Suwanee, GA (US)

Correspondence Address: BIOCURE, INC., 2975 GATEWAY DRIVE, SUITE 100, NORCROSS, GA, 30071, US

(21) Appl. No.: 09/960,449

(22) Filed: Sep. 21, 2001

Related U.S. Application Data
(60) Provisional application No. 60/235,168, filed on Sep. 23, 2000.

(51) Int. Cl. A61K 38/43; A61K 38/18; A61K 33/00; A61K 9/00

(52) U.S. Cl. 424/43; 514/2; 424/94.1; 424/718

(57) ABSTRACT

A wound dressing is provided that is formed in situ on the wound by spray application of a composition including a composition that gels in situ to form a hydrogel. The composition preferably includes macromers having water soluble regions and crosslinkable regions. Compositions for making a hydrogel wound dressing are also provided.

This application claims priority to U.S. Provisional application Serial No. 60/235,168, filed on Sep. 23, 2000.

### FIELD OF THE INVENTION

The present invention is generally in the field of wound dressings. More specifically, the present invention is directed to hydrogel wound dressings that can be sprayed onto a wound as a liquid, and then crosslink or otherwise thicken to form a hydrogel in situ.

### BACKGROUND OF THE INVENTION

Wound dressings can be used both to protect and to enhance the healing of a wound. Desirable characteristics of wound dressings vary depending upon the type of wound to be treated but generally include that they be moist, conformable to the wound topography (flexible), sterile, and adherent to the surrounding tissue but able to be easily removed from the surrounding tissue as well as the wound. In may be desirable in some cases for the dressing to remove exudates from the wound. It may be desirable in the case of some wounds to debride the wound, which can be accomplished using a dressing that adheres to the wound surface, so that the wound is debrided as the dressing is removed from the wound.

Many different types of wound dressings have been developed. However, most of these dressings have had one or more disadvantages, including the need for frequent removal, difficulty in adhesion, improper mechanical properties, or difficulty in application. Most currently available topical therapeutic formulations are applied as an ointment, cream, or liquid and are used in combination with a wound covering, or bandage. Whether used with or without a covering, a topical formulation in the form of a cream, ointment, or liquid is difficult to apply and maintain at the injury site. It can be rapidly removed by mechanical action and/or dissolution by body fluid. If used in combination with a covering, therapeutic formulations often have several other drawbacks including lack of biodegradability, damage or irritation to the skin during removal of the covering, covalent bonding or other interaction of the therapeutic agent and the covering, inability to use a wide variety of therapeutic agents, and inadequate adhesion of the covering.

Hydrogel wound dressings have been developed that are applied to the wound either as a pre-formed solid gel or as a liquid that is poured onto the wound and that gels after application. Application of a composition as a gellable liquid offers a potential advantage over a pre-formed solid gel in that it will result in a dressing that conforms to the surface of the wound. However, such formulations have several disadvantages, including that the composition does not stay in place when applied as a poured-on liquid.

Wound dressings are used to accelerate healing of all types of wounds, including burns, surgical wounds, and open leg and foot wounds. There are generally three types of open leg wounds, termed ulcers: venous stasis ulcers, generally seen in sedentary elderly people when blood flow to the leg becomes sluggish; decubitus ulcers, also termed pressure sores or bed sores, which occurs most often in people who are bedridden and are unable to frequently change position; and diabetic foot ulcers, caused by poor blood circulation to the feet. Due to the aging of the population, there will likely be a greater demand for effective and user friendly wound treatments in the near future.

It would be advantageous to have a wound dressing that is easy to apply and remove in addition to performing the desirable functions of a wound dressing, such as providing moisture and protection, that is conformable and adherent to the surrounding tissue.

It would be advantageous to have a wound dressing that delivers a therapeutic formulation directly to the wound and that also performs the desirable functions of a wound dressing, such as providing moisture and protection, that is conformable and adherent to the surrounding tissue.

### SUMMARY OF THE INVENTION

A composition is provided that forms a dressing in situ on a wound. The liquid composition is sprayed onto the wound, whereupon it polymerizes or otherwise thickens to form a hydrogel wound dressing.

The method of forming a wound dressing is fast, clean, and simple. A single embodiment of the spray on wound dressing can be applied to both small and large wounds using the same device, thereby reducing the need of carrying a large inventory of different sized pre-formed dressings. Spray delivery can increase the penetration of the polymer into the wound area thereby potentially making the delivery of active ingredients more efficient. Penetration of the polymer into the wound bed may also aid in debridement of the wound during dressing changes to accelerate the wound healing process.

The composition can be designed for use with wounds having certain characteristics. For example, wounds having a lot of exudate can be dressed using a composition including an absorbent. A wound needed to be debrided can be dressed using a composition that is more adherent to the wound.

### DETAILED DESCRIPTION OF THE PREFERRED EMBODIMENTS

Definitions

The term “wound” as used herein refers to all types of tissue injuries, including those inflicted by surgery and trauma, including bums, as well as injuries from chronic or acute medical conditions, such as atherosclerosis or diabetes. The compositions and wound dressings described herein are useful for treatment of all types of wounds, including wounds to internal and external tissues.

The term “hydrogel” as used herein refers to a material having an aqueous phase with an interlaced polymeric component, with at least 10% to 90% of its weight as water.

The term “biodegradable” as used herein refers to materials that are non-permanent and removed by natural or imposed biological and/or chemical processes.

The term “spray” as used herein refers to an atomized composition, such as comprised of small or large liquid droplets, such as applied through an aerosol applicator or pump spray applicator.

Wound dressings containing a hydrogel are disclosed. Compositions useful for forming hydrogel wound dressings are disclosed. The compositions include a macromolecular monomer (termed herein a “macromer”) or polymer that gels or otherwise thickens in situ to form a hydrogel.

Hydrogels can be formed in situ on the wound surface using a variety of methods. The composition is applied as a pre-gelled formulation of monomers, macromers, polymers, or combinations thereof, maintained as solutions, suspensions, or dispersions, referred to herein jointly as “solutions” unless otherwise stated, that forms the hydrogel upon or shortly after application.

The compositions are applied to a wound by a spray, such as via a pump or aerosol device. In one embodiment, a solution, dispersion, or suspension of a composition is sprayed onto the wound to form the wound dressing. A stimulus is brought into contact with the pre-gelled composition, before, during, or after application of the composition to the wound, causing crosslinking or other thickening of the macromer or polymer to form the hydrogel. For example, the gelling stimulus could be light in the form of ambient light or a light wand that is passed over the wound, or could be a redox pair, each of which can initiate free radical crosslinking of unsaturated end groups on macromers. In one embodiment, the composition includes macromers capable of crosslinking to form the hydrogel. The macromers are sprayed onto the wound and are crosslinked in situ to form the hydrogel wound dressing. Other compositions that can be used include polymers that polymerize or otherwise thicken in response to temperature or pH change, and macromers that crosslink by other mechanisms. For example, the pre-gelled polymeric solution can contain a certain amount of volatile solvent that evaporates upon application, causing the polymer to precipitate into a hydrogel. A pre-gelled polymeric solution could alternatively be designed to gel in response to an environmental change, such as a change in temperature or pH, or upon contact with a stimulus, such as blood.

The hydrogel dressing protects the wound, functions in wound healing, and may be used as a drug delivery system to provide an active agent to the wound.

I. The Compositions

A. General Characteristics of the Hydrogel Wound Dressings

The in situ formed dressing should be conformable and compliant so that it conforms to the topography of the wound and the tissue surface around the wound and is comfortable to wear. Conformability will also extend the longevity of the dressing. The dressing is also preferably strong enough that it can be peeled off of the wound.

In some cases, such as acute wounds, it is desirable to debride the wound. In other cases, such as chronic wounds, debridement is not desirable. The amount of debridement facilitated by the dressing is, in part, dependent on the degree to which the gel adheres to the wound surface. Greater adherence can be achieved by including a more adherent monomer or polymer, such as a hydrophobic monomer or polymer, in the composition. Alternatively, an ingredient can be included in the composition formulation that provides adherence to the wound.

The dressing is preferably nondegradable, although there may be situations where it may be desirable for the dressing to be biodegradable. For example, it may be desirable in some cases to apply a very thin hydrogel coating that will degrade and be removed from the wound in a few days.

The dressing should be sterile so it must be possible to achieve and maintain sterility of the composition and deliver the composition so that sterility is maintained.

It may be desirable for the wound dressing to absorb some or a large amount of exudates from the wound. The amount of moisture that can be absorbed by the wound dressing can be manipulated by adjusting the composition to include more of less of an absorbent monomer. For example, noncrosslinked hydrophilic monomers or polymers, such as, but not limited to, polyvinyl alcohols (PVA), polyethylene glycols (PEG), or polyvinyl pyrrolidone (PVP), can be included in the composition. The absorption capability of the dressing can also be increased by including an absorbent in the composition, as discussed more below.

B. Components of the Compositions

The compositions include one or a combination of monomers, macromers, and/or polymers that polymerize or otherwise thicken upon or shortly after delivery, to form a hydrogel dressing on a wound. The compositions further desirably include a solvent, desirably aqueous, and may include additives, such as an absorbent, and one or more active agents. All of the composition ingredients should be biocompatible or non-irritant in the amounts present in the final hydrogel dressing.

The viscosity of the pre-gelled composition should be great enough so that the solution stays in place before gelling occurs. The viscosity should be low enough that the composition can be sprayed using the spray delivery device. Appropriate viscosity depends upon the molecular weight of the composition monomers, macromers, and/or polymers, and the delivery means to be employed. Generally, the composition should have a viscosity lower than about 400 cps, preferably lower than 300 cps, more preferably lower than 200 cps to be delivered via aerosol. Delivery through a pump spray generally requires a lower viscosity, such as less than about 150 cps.

Gelling of the pre-gelled composition on the wound is preferably rapid, to avoid run off of the composition from the place of application. The gelling time can be 5 minutes or less, preferably less than about three minutes, more preferably less than about 1 minute, and, in some situations, as low as about 10 seconds or less. A composition that is more adherent to the wound surface could reasonably have a longer gelation time.

Macromer Systems

The hydrogel can be made from one or more macromers. Macromers include a hydrophilic or water soluble region and one or more crosslinkable regions. The macromers may also include other elements such as one or more degradable or biodegradable regions. A variety of factors—primarily the desired characteristics of the formed hydrogel dressing—determines the most appropriate macromers to use. The basic requirements for the macromers are biocompatibility and the capacity to be applied in spray to the wound whereupon it forms a gel. Many macromer systems that form biocompatible hydrogels can be used.

Macromers can be constructed from a number of hydrophilic polymers, such as, but not limited to, polyvinyl alcohols (PVA), polyethylene glycols (PEG), polyvinyl pyrrolidone (PVP), polyalkyl hydroxy acrylates and methacrylates (e.g. hydroxyethyl methacrylate (HEMA), hydroxybutyl methacrylate (HBMA), and dimethylaminoethyl methacrylate (DMEMA)), polysaccharides (e.g. cellulose, dextran), polyacrylic acid, polyamino acids (e.g. polylysine, polyethyimine, PAMAM dendrimers), polyacrylamides (e.g. polydimethylacrylamid-co-HEMA, polydimethylacrylamid-co-HBMA, polydimethylacrylamid-co-DMEMA). The macromers can be linear or can have a branched, hyperbranched, or dendritic structure.

The macromers include two or more crosslinkable groups. Crosslinking of macromers may be via any of a number of means, such as physical crosslinking or chemical crosslinking. Physical crosslinking includes, but is not limited to, complexation, hydrogen bonding, desolvation, Van der waals interactions, and ionic bonding. Chemical crosslinking can be accomplished by a number of means including, but not limited to, chain reaction (addition) polymerization, step reaction (condensation) polymerization and other methods of increasing the molecular weight of polymers/oligomers to very high molecular weights. Chain reaction polymerization includes but is not exclusive to free radical polymerization (thermal, photo, redox, atom transfer polymerization, etc.), cationic polymerization (including onium), anionic polymerization (including group transfer polymerization), certain types of coordination polymerization, certain types of ring opening and metal metathesis polymerizations, etc. Step reaction polymerizations include all polymerizations which follow step growth kinetics including but not limited to reactions of nucleophiles with electrophiles, certain types of coordination polymerization, certain types of ring opening and metal metathesis polymerizations, etc. Other methods of increasing molecular weight of polymers/oligomers include but are not limited to polyelectrolyte, formation, grafting, ionic crosslinking, etc.

In one embodiment, the hydrogel is formed from macromers having a backbone of a polymer comprising units having a 1,2-diol or 1,3-diol structure, such as polyhydroxy polymers. For example, polyvinyl alcohol (PVA) or copolymers of vinyl alcohol contain a 1,3-diol skeleton. The backbone can also contain hydroxyl groups in the form of 1,2-glycols, such as copolymer units of 1,2-dihydroxyethylene. These can be obtained, for example, by alkaline hydrolysis of vinyl acetate-vinylene carbonate copolymers. Other polymeric diols can be used, such as saccharides.

The macromers have at least two pendant chains containing groups that can be crosslinked. The term group includes single polymerizable moieties, such as an acrylate, as well as larger crosslinkable regions, such as oligomeric or polymeric regions. The crosslinkers are desirably present in an amount of from approximately 0.01 to 10 milliequivalents of crosslinker per gram of backbone (meq/g), more desirably about 0.05 to 1.5 meq/g. The macromers can contain more than one type of crosslinkable group.

The pendant chains are attached via the hydroxyl groups of the polymer backbone. Desirably, the pendant chains having crosslinkable groups are attached via cyclic acetal linkages to the 1,2-diol or 1,3-diol hydroxyl groups.

In one preferred embodiment, the compositions include modified polyvinyl alcohol (PVA) macromers, such as those described in U.S. Pat. Nos. 5,508,317, 5,665,840, 5,849,841, 5,932,674, 6,011,077, 5,939,489, or 5,807,927. The macromers disclosed in U.S. Pat. No. 5,508,317, for example, are PVA prepolymers modified with pendant crosslinkable groups, such as acrylamide groups containing crosslinkable olefinically unsaturated groups. These macromers can be polymerized by photopolymerization or redox free radical polymerization, for example.

The hydrophobicity of these macromers can be increased by substituting some of the pendant hydroxyl groups with more hydrophobic substituents. The properties of the macromers, such as hydrophobicity, can also be modified by incorporating a comonomer in the macromer backbone. The macromers can also be formed having pendant groups crosslinkable by other means.

If a degradable hydrogel is desired, the macromers disclosed in WO 01/44307 can be used. Other suitable macromers include those disclosed in U.S. Pat. No. 5,410,016 to Hubbell et al., U.S. Pat. No. 4,938,763 to Dunn et al., U.S. Pat. No. 5,100,992 and U.S. Pat. No. 4,826,945 to Cohn et al., U.S. Pat. Nos. 4,741,872 and 5,160,745 to De Luca et al, and U.S. Pat. No. 4,511,478 to Nowinski et al.

In one preferred embodiment, the macromers are PEG diacrylates.

In one embodiment of the composition, wherein the macromers have free radical polymerizable groups, a two part redox system is employed. One part of the system contains a reducing agent such as ferrous salt. Various ferrous salts can be used, such as ferrous gluconate dihydrate, ferrous lactate dihydrate, or ferrous acetate. The other half of the solution contains an oxidizing agent such as hydrogen peroxide. Either or both of the redox solutions can contain macromer, or it may be in a third solution. The two solutions are sequentially or simultaneously applied to a wound using an aerosol or pump action spray applicator. The agents react to initiate the polymerization of the macromer to generate a crosslinked hydrogel coating.

Other reducing agents can be used, such as, but not limited to cuprous salts, cerous salts, cobaltous salts, permanganate, and manganous salts. Other oxidizing agents that can be used include, but are not limited to, t-butyl hydroperoxide, t-butyl peroxide, benzoyl peroxide, cumyl peroxide, etc.

In another embodiment, a single component system is utilized. A solution containing the macromer is mixed with a UV photoinitiator such as Irgacure 2959. The solution can be applied to a wound using an aerosol or pump spray applicator then irradiated with UV light to generate a crosslinked hydrogel coating. With the use of a suitable photoinitiator, visible light may be used to initiate polymerization

In another embodiment, the macromer solution is complexed in situ with borate ions to generate a hydrogel. A solution of macromer and a borate solution are simultaneously or sequentially applied to a wound using an aerosol or pump spray applicator to generate a polymer film.

Polymer Systems

Other compositions can be used that include polymers that polymerize or otherwise thicken in response to temperature or pH change. For example, a polymeric solution can contain a certain amount of volatile solvent that evaporates upon application, causing the polymer to precipitate into a hydrogel. A polymeric solution could alternatively be designed to gel in response to an environmental change, such as a change in temperature or pH, or upon contact with a stimulus, such as blood. For example, PVA can be modified with hydrophobic groups rendering it soluble in a water/organic solvent, but insoluble when the organic solvent evaporates or is diluted in a water based environment such as a wound.

The composition can include a combination of macromers and polymers to achieve a final hydrogel having the desired properties.

The composition can be supplied in an aqueous solution or it may be preferable to include all or a portion of nonaqueous solvent to solubilize the macromer or polymer or another additive of the composition. A biocompatible solvent should be used. It may be desirable to use a volatile solvent so that the viscosity of the composition is quickly decreased after it is applied to the wound, to hasten gelling and to minimize run off. Any solvent that is compatible with the other elements of the composition and that is not harmful to the tissue being treated can be used. Examples include ethanol and isopropanol. Other solvents can be used in small amounts to solubilize components, such as DMSO and N-methyl pyrrolidone.

Propellants

If the composition is supplied as an aerosol, a propellant is used to provide the force for expulsion of the composition from the container. It is desirable that the macromeric or polymeric composition and the propellant form a single liquid phase so that the composition is delivered consistently.

Any of a number of propellants known to those skilled in the art can be used, provided that it is chemically inert to the other ingredients of the composition. Suitable propellants include vinyl chloride and mixtures of vinyl chloride and dichlorodifluoromethane, other fluorochlorohydrocarbons known as the Freons and the Genetrons, and blends of fluorochlorohydrocarbons, chlorinated hydrocarbons, and hydrocarbons. Examples of fluorochlorohydrocarbons include trichloromonofluoromethane, dichlorodifluoromethane, dichloromonofluoromethane, 2-tetrafluoroethane, 1,1-dichloro-1,2,2-tetrafluoroethane, 1-chloro-1,1-difluoroethane, 1,1-difluoroethane, and octofluorocyclobutane, and mixtures thereof. Examples of hydrocarbons include liquefied petroleum gases like propane, isobutane, and N-butane and mixtures thereof. Dimethyl ether is another propellant. Compressed gas propellants that are preferably non-toxic, non-flammable, and inert can be used. Examples include carbon dioxide, nitrous oxide and N₂ and the like. Mixtures of the above are often used.

The quantity of propellant used is critical only in that if an insufficient amount is used, the driving force to expel the entire composition from the container will be lacking. Generally, the composition will comprise from 75% to 95% by weight propellant.

Aerosol devices such as the Preval® aerosol spray unit available from Precision Valve Corporation, NY, USA, can be used. This device has a modular power unit and refillable container jar. The propellant is a mixture of propane, isobutane, and dimethyl ether.

Active Agents

The dressing may function as a drug delivery matrix to deliver active agents to the wound. Biologically active agents that it may be desirable to deliver include prophylactic, therapeutic, and diagnostic agents (collectively referred to herein as “active agent” or “drug”). A wide variety of bioactive agents can be incorporated into the hydrogel. Release of the incorporated additive from the hydrogel to the wound is achieved by diffusion of the additive from the hydrogel, degradation of the hydrogel, and/or degradation of a chemical link coupling the agent to the polymer.

Active agents include, but are not limited to, agents to promote tissue healing, agents to promote sterility, agents to reduce pain, and agents to promote tissue growth. Active agents that can be added include, for example, growth factors (e.g. platelet-derived growth factor, epidermal growth factor, transforming growth factor beta (TGF-β)), nitric oxide, antibiotics, silver, anti-inflammatories, analgesics, blood coagulants, and enzymes.

Cells can be included in the wound dressing to encourage tissue growth. The cells can be living (whether naturally occurring or produced through recombinant DNA technology), artificial cells, cell ghosts (i.e. RBC or platelet ghosts), or pseudovirions, to serve any of several purposes. For example, the cells may be selected to produce specific agents such as growth factors at the wound location.

Release of the incorporated additive from the hydrogel is achieved by diffusion of the agent from the hydrogel, degradation of the hydrogel, and/or degradation of a chemical link coupling the agent to the polymer. In this context, an “effective amount” refers to the amount of active agent required to obtain the desired effect.

Active agents can be incorporated into the hydrogel dressing simply by mixing the agent with the composition prior to or upon administration. The active agent will then be entrapped in the hydrogel that is formed upon administration of the composition. The active agent can be in compound form or can be in the form of degradable or nondegradable nano or microspheres. It some cases, it may be possible and desirable to attach the active agent to the macromer. The active agent may be released from the macromer or hydrogel over time or in response to an environmental condition. The active may be attached by a degradable linkage, such as a linkage susceptible to degradation via hydrolysis or enzymatic degradation. The linkage may be one which is susceptible to degradation at a certain pH, for example. The active agent can be encapsulated in liposomes, which are then entrapped in the hydrogel.

The only limitation as to how much active agent(s) can be loaded into the compositions is one of functionality, namely, the drug load may be increased until the crosslinking of the macromers is adversely affected to an unacceptable degree, or until the properties of the formulation are adversely affected to such a degree as to make administration of the formulation unacceptably difficult. Generally speaking, it is anticipated that in most instances the active agent will make up between about 0.01 to 20% by weight of the formulation with ranges of between about 0.01 to 10% being highly common. These ranges of drug loading are not limiting to the invention. Provided functionality is maintained, drug loadings outside of these ranges fall within the scope of the invention.

The compositions can also be applied over an active agent—the active agent can be applied in the form of a powder or gel, for example, and then the composition can be sprayed over the active agent.

Other Components of the Compositions

The composition may additionally contain one or more additives such as preservatives, defoamers, pore forming agents, plasticizers, penetration enhancers, colorants, wettings agents, leveling agents, hydrating agents, thickeners, fillers, opacifying agents, and absorbents. Any additive should be biocompatible and should be able to be delivered through the spray delivery device.

Defoamers include, for example, Agitan 290 and Colloid 513, added in an amount effective to reduce the amount of air bubbles in the sprayed gel and facilitate spray application.

Wetting and leveling agents include, for example, Pluronics surfactants and fluorinated Zonyl surfactants. Such agents can facilitate spreading of the sprayed composition evenly on the wound surface.

Hydrating agents include, for example, glycerol and polyvinyl pyrrolidone and may be added to the sprayed solution in order to maintain hydration of the wound dressing.

Thickeners and fillers can be added to aid in application (reduce run off) and include, for example, polyacrylate (Carbopol from B. F. Goodrich) and other thixotropic agents.

Absorbents can be added to increase absorption of exudates from the wound and include, for example, polyacrylic acid, starch, celluloses such as carboxymethylcelluose, calcium alginate (which also may act to control bleeding), sugars (sorbitol, mannitol, zylitol), glycerin, dextrans, and hyaluronic acid.

The additive can be simply mixed with the macromer composition, can be chemically or physically coupled to the macromer, or can be provided in a delivery vehicle such as controlled release microcapsules or liposomes that may provide controlled release of the additive. The method of chemically coupling an additive to the macromer will be dependent upon the chemical nature of the additive and the macromer. Coupling chemistries are known to those skilled in the art and can be readily designed by one skilled in the art.

Nitric oxide can be in the form of nitric oxide precursors bound to or otherwise incorporated into the hydrogel. Examples include complexes of NO with nucleophiles (termed diazeniumdiolates or NONOates) as discussed by Keefer et al. in U.S. Pat. Nos. 5,405,919 and 5,718,892. Other examples include sodium nitroprusside, S-nitrosothiols (Diodati et al. 1993), nitrate esters, t-organonitroso compounds, organic nitrates (Ignarro et al. 1981), inorganic nitrites (see U.S. Pat. No. 5,994,444 to Trescony et al.), nitrosated amines, O-alkylated diazeniumdiolates, nitric oxide prodrugs, etc. In addition, L-arginine increases endogenous NO production. The prior art teaches methods of attaching many of these compounds to the backbone of the hydrogel polymers.

II. Delivery of the Compositions

The composition is delivered to the wound from a spray device. The spray device includes a container having a dispenser for spray delivery of the liquid composition. The type of container used is variable, depending upon compatibility with the composition and the spray dispenser and can be glass, plastic, metal, etc. Generally, any chemical, mechanical or electronic method for propelling the liquid composition as a spray from the container is appropriate. In one embodiment, a compatible liquid or gaseous aerosol propellant is placed in an appropriate container along with the composition and the dispenser includes a valve mechanism that enables atomized spray delivery of the liquid composition.

In general, for aerosol delivery, the ingredients of the composition are mixed to form a substantially homogenous solution, slurry, dispersion, or the like. For two part systems, each part is mixed. The compositions are placed in an appropriate container and the propellant is added using conventional techniques, such as cold filling or pressure filling techniques. The composition can be delivered using a commercially available aerosol sprayer such as, for example, the Preval® aerosol spray unit available from Precision Valve Corporation, NY, USA, which includes a propellant unit having attached thereto a container for the composition.

The composition can also be delivered using a syringe outfitted with a spray head, or a dual spray device outfitted with a spray head and, optionally, a mixing chamber.

For two part systems, a device should be used having two containers so that the components are kept apart until use. A device having a single dispenser can be used, or a device having a double dispenser can be used. If a double dispenser is used, the sprays from the dispensers can be aligned to substantially overlap. A suitable device is described in U.S. Pat. No. 5,989,215, for example. It is also possible, although less preferred, to apply the two parts sequentially. A mixer may be employed in the case of a single dispenser to mix the two parts before or during spraying.

The device may include a meter so that the quantity of composition can be controlled.

III. Methods of Using the Compositions

The pre-gelled composition is applied to the wound as a spray using an appropriate delivery device. The composition should be applied to result in a hydrogel having a thickness ranging from about 0.001 to 5 mm, desirably about 0.01 to 2.5 mm. It may be desirable to apply several layers of the pre-gelled composition to the wound to ensure adequate coverage of the wound.

The dressing can be covered with a secondary dressing, or bandage, if desired to protect the hydrogel or to provide additional moisture absorption, for example. It may be advantageous to apply the dressing using a drape to define the area to be treated.

If desirable, the dressing is removed after a period of time, the wound can be cleaned if desired, and a new dressing can be applied. It may be desirable to apply compositions having different formulations at different stages of wound healing.

The dressings can be used on all types of wounds, with appropriate modification of the formulation, as discussed above. The compositions can be applied to skin, mucous membranes, body cavities, and to internal surfaces of bones, tissues, etc. that have been damaged. The dressings can be used on wounds such as cuts, abrasions, ulcers, surgical incision sites, burns, and to treat other types of tissue damage.

The compositions can be applied over an active agent—the active agent can be applied in the form of a powder or gel, for example, and then the composition can be sprayed over the active agent. The compositions could also be applied over skin substitutes, such as Apligraf®, a product by Novartis, and other products.

The examples below serve to further illustrate the invention, to provide those of ordinary skill in the art with a complete disclosure and description of how the compounds, compositions, articles, devices, and/or methods claimed herein are made and evaluated, and are not intended to limit the scope of the invention. In the examples, unless expressly stated otherwise, amounts and percentages are by weight, temperature is in degrees Celsius or is at ambient temperature, and pressure is at or near atmospheric. The examples are not intended to restrict the scope of the invention.

### EXAMPLES

### Examples 1-3

Two Part Redox Systems

The following examples employed two part redox initiated hydrogels. Ascorbic acid was used as a 415 mM solution. Hydrogen peroxide was used as a 415 mM solution. Ferrous acetate was prepared as a 41.5 mM solution.

The PVA macromer used in examples 1-3 had a Mw of 37,000, tradename Mowiol 5-88 from Hoechst AG, Germany. The PVA was modified with 0.20 meq/g of N-acryloly aminoacetaldehyde dimethylacetal (NAAADA) as described in U.S. Pat. No. 5,508,317 to Muller et al. The PVA macromer was used as a 20% solution in water. Each of the solutions, containing reductant and oxidant, contained macromer.

Table 1 shows the compositions used for Examples 1-3.

TABLE 1

| Example | 1 | | 2 | | 3 | |
|---|---|---|---|---|---|---|
| Solution | A | B | A | B | A | B |
| PVA (g) | 40 | 40 | 40 | 40 | 40 | 40 |
| Water (ml) | 20 | 20 | 20 | 20 | 20 | 20 |
| Fe Solution (ml) | 2.40 | — | 3.60 | — | 4.80 | — |
| Ascorbic Acid Solution (ml) | 1.92 | 1.92 | 1.92 | 1.92 | 1.92 | 1.92 |
| Acetate Buffer (1 M, pH 4.2) | 1.92 | 1.92 | 1.92 | 1.92 | 1.92 | 1.92 |
| (ml) | | | | | | |
| Peroxide Solution (ml) | — | 1.92 | — | 2.86 | — | 3.80 |

Each half of the redox pair was placed into a separate 100 ml glass amber bottle which was screwed onto a Preval® aerosol spray unit (Precision Valve Corporation, NY, USA). Each redox pair was simultaneously sprayed on a single spot against a vertical quartz glass surface for approximately 10 seconds. The macromers quickly crosslinked into a polymer film. The polymer film could be peeled or washed of off the glass with a stream of water. The speed of crosslinking increased as the concentration of the redox initiator was increased, and the amount of run-off prior to the onset of gelation was reduced. Example 3 was re-applied to the vertical glass surface in five two second bursts (5×2 sec) instead of as a continuous application to determine whether a uniform film would be formed. The polymer film that formed in each of the examples was strong enough that it could be peeled off the surface or washed off with a stream of water. The polymer film formed in Example 3 appeared similar to those from Examples 1 and 2.

### Example 4

Addition of Surfactant

Example 3 was repeated with the addition of 0.5 g of Zonyl FS-300 (DuPont) to each half of the redox pair. This fluorinated surfactant can act as a leveling agent. Each half of the redox solution was simultaneously sprayed against a vertical glass surface. The polymer film that formed was strong enough to be peeled off the surface or washed off with a stream of water.

### Example 5

Addition of a Hydrating Agent

Example 4 was repeated with the addition of 0.5 g of PVP/W-635 (a PVP/vinyl acetate copolymer from ISP Technologies, Wayne, N.J., USA) to each half of the redox pair. This hydrophilic copolymer is well known to be a hydrating agent. Each half of the redox solution was simultaneously sprayed against a vertical glass surface. The polymer film that formed was strong enough that it could be peeled off the surface or washed off with a stream of water.

### Example 6

Using a Hand Pump Sprayer

The redox pair of example 5 was applied to the vertical surface of a quartz glass plate via a hand pump spray applicator. A crosslinked polymer quickly developed. The polymer film could be peeled or washed off the glass with a stream of water.

### EXAMPLES 7 and 8

Limited Dilution

The PVA macromer used in examples 7 and 8 was the same as that used in Examples 1 through 6. Table 2 displays the compositions used fro Examples 7 and 8.

TABLE 2

| | | |
|---|---|---|
| Example | 7 | 8 |

| Solution | A | B | A | B |
|---|---|---|---|---|
| PVA (g) | 30 | 30 | 12 | 12 |
| Water (ml) | — | — | 12 | 12 |
| Fe Solution (ml) | 2.40 | — | 2.00 | — |
| Ascorbic Acid Solution (ml) | 1.92 | — | 1.60 | — |
| Acetate Buffer (1M, pH 4.2) (ml) | 1.20 | 1.20 | 0.50 | 0.50 |
| Peroxide Solution (ml) | — | 1.92 | — | 1.60 |

Example 7 was prepared as less diluted solutions in an attempt to minimize or eliminate any run off prior to gelation of the composition. Each half of the redox pair was placed into a separate 100 ml glass amber bottle which were then screwed onto a Preval® aerosol spray unit. Each redox pair was simultaneously sprayed on a single spot against a vertical quartz glass surface for approximately 10 seconds. A crosslinked polymer quickly developed and there was less run off. The polymer film could be peeled off or washed off the glass with a stream of water.

### Examples 9 through 11

Borate as Initiator and Addition of Hydrating Agent

PVA macromer used in examples 8-10: Mowiol 5-88 (Mw=37,000) modified with 0.25 meq/g of N-acryloly aminoacetaldehyde dimethylacetal (NAAADA). The PVA macromer was used as a 20% solution in water. Table 3 shows the compositions used for Examples 9-11.

TABLE 3

| Example | 9 | | 10 | | 11 | |
|---|---|---|---|---|---|---|
| Solution | A | B | A | B | A | B |
| PVA (g) | 40 | — | 40 | — | 40 | — |
| Water (ml) | 10 | 20 | 10 | 20 | 10 | 20 |
| Fe Solution (ml) | — | — | — | — | 1.20 | — |
| Ascorbic Acid Solution (ml) | — | — | — | — | 0.96 | — |
| Acetate Buffer (1 M, pH 4.2) | — | — | — | — | 1.20 | — |
| (ml) | | | | | | |
| Peroxide Solution (ml) | — | — | — | — | — | 0.96 |
| 5% Borate (ml) | — | 20 | — | 20 | — | 20 |
| PVP copolymer (g) | — | — | — | 10 | — | 10 |

Examples 9 and 10 highlight the ability of the sprayed PVA macromer to be crosslinked by complex formation with borate ions in place of a redox cure mechanism. Each solution pair was placed into separate 100 ml glass amber bottle then screwed onto a Preval® aerosol spray unit. Each solution was simultaneously sprayed on a single spot against a vertical quartz glass surface for approximately 10 seconds. The resulting polymer was soft and very elastic and could be removed from the surface by wiping. Examples 10 and 11 included the addition of PVP/VAW-635 as a potential hydrating agent for the crosslinked film. The resulting polymer film was similar to that of Example 9.

Example 11 combined redox curing with complex formation of borate ions. Each solution pair was placed into separate 100 ml glass amber bottle then screwed onto a Preval® aerosol spray unit. Each solution was simultaneously sprayed on a single spot against a vertical quartz glass surface for approximately 10 seconds. The resulting polymer was soft and very elastic.

### Examples 12 and 13

Photopolymerization

PVA macromer used in examples 11-12: Mowiol 3-83 (Mw=14,000) modified with 0.45 meq/g of N-acryloly aminoacetaldehyde dimethylacetal (NAAADA). The PVA macromer was used as a 15% solution in water and contained 0.1% w/w of the photoinitiator Irgacure 2959. Table 4 shows the compositions used for Examples 12 and 13.

TABLE 4

| Example | 12 | 13 |
|---|---|---|
| PVA (g) | 40 | 40 |
| Triethanolamine | — | 1.2 |

Examples 12 and 13 demonstrate the ability of the sprayed PVA solution to be cured by UV photoinitiated polymerization. The solution was placed in to a glass amber bottle and attached to a Prevalo® aerosol applicator. The PVA solution was sprayed into a plastic petri dish then cured under a UV lamp (3 mW/cm⁻², 310 nm) for twenty seconds. The resulting polymer could be peeled off the petri dish with a pair of tweezers.

### Example 14

Application to Chicken Skin and a Human Hand

PVA macromer used in example 13: Mowiol 4-88 (Mw=31,000) modified with 0.036 meq/g of N-acryloly aminoacetaldehyde dimethylacetal (NAAADA). Table 5 shows the compositions used for Example 14.

TABLE 5

| | |
|---|---|
| Example | 14 |

| Solution | A | B |
|---|---|---|
| PVA (g) | 25 | 25 |
| Water (ml) | 15.40 | 20.40 |
| Fe Solution (ml) | 5.00 | — |
| Ascorbic Acid Solution (ml) | 4.00 | — |
| Acetate Buffer (1M, pH 4.2) (ml) | 0.60 | 0.60 |
| Peroxide Solution (ml) | | 4.00 |

Each half of the redox pair was placed into separate 100 ml glass amber bottle then each screwed onto a Preval® aerosol spray unit. Each redox pair was simultaneously sprayed on a chicken skin attached to vertical quartz glass surface for approximately 10 seconds. A crosslinked polymer quickly developed. The polymer film could be peeled off or washed off the chicken skin with a stream of water. The composition was also applied vertically to the wet and dry back of a person's hand for approximately 10 seconds. The polymer film that formed was adherent to the skin and did not fall off the skin when the hand was shaken. The film peeled off more easily from the wet hand than the dry hand.

### Example 15

More Hydrophobic Composition

The PVA macromer used in Example 15 was Mowiol 4-88 (Mw=31,000) modified with 0.09 meq/g of N-acryloly aminoacetaldehyde dimethylacetal (NAAADA) and 4 meq/g of acetaldehyde dimethyl acetal. The PVA macromer was used as a 20% solution in water. Table 6 shows the compositions used for Example 15.

TABLE 6

| | |
|---|---|
| Example | 15 |

| Solution | A | B |
|---|---|---|
| PVA (g) | 25 | 25 |
| Water (ml) | 15.40 | 20.40 |
| Fe Solution (ml) | 5.00 | — |
| Ascorbic Acid Solution (ml) | 4.00 | — |
| Acetate Buffer (1M, pH 4.2) (ml) | 0.60 | 0.60 |
| Peroxide Solution (ml) | | 4.00 |

Each half of the redox pair was placed into a separate 100 ml glass amber bottle and then each was screwed onto a Preval® aerosol spray unit. Each redox pair was simultaneously sprayed on a chicken skin attached to a vertical quartz glass surface for approximately 10 seconds. A crosslinked polymer quickly developed. The polymer film could be peeled off or washed off the chicken skin with a stream of water. The composition was also applied vertically to the wet and dry back of a person's hand for approximately 10 seconds. The polymer film that formed was adherent to the skin and did not fall off the skin when the hand was shaken. The film peeled off more easily from the wet hand than the dry hand.

### Example 16

Application Using a Dual Syringe Spray

The PVA macromer used in example 16 was Mowiol 5-88 (Mw=37,000) modified with 0.25 meq/g of N-acryloly aminoacetaldehyde dimethylacetal (NAAADA). The PVA macromer was used as a 20% solution in water. Table 7 shows the compositions used for Example 16.

TABLE 7

| | |
|---|---|
| Example | 16 |

| Solution | A | B |
|---|---|---|
| PVA (g) | 20 | 20 |
| Water (ml) | 13.40 | 16.60 |
| Fe Solution (ml) | 3.20 | — |
| Ascorbic Acid Solution (ml) | 2.60 | — |
| Acetate Buffer (1M, pH 4.2) (ml) | 0.80 | 0.80 |
| Peroxide Solution (ml) | | 2.60 |

The redox solutions were loaded into a Fibrijt 5 cc dual syringes fitted with a Fibrijet dual syringe spray applicator (Micromedics Inc, Egan, Minn.). The solution was applied to a glass surface to form a crosslinked polymer. Diluting the redox solutions from 10 to 8 weight % dramatically improved the spray characteristics of the solutions to generate polymers which could be peeled off the glass.

### Example 17

PEG Macromers

The various types of PEG diacrylates prepared were 4.6KPEGA2 (4.6 KDa PEG having acrylate end caps), 5KPEGA2, 10KPEGA2, 20KPEGA2, and 35KPEGA2. These macromers were made as described in literature. Small amounts of the comonomer vinylcaprolactam (VC) were added to some formulations to decrease swellability of the formed hydrogels.

Between various PEG macromers, the PEGs that have a greater molecular weight between crosslinks (such as 20KPEGA2 or 35KPEGA2) yielded softer, more flexible films than those prepared from lower molecular weight PEGs such as 4.6KPEGA2 and 10KPEGA2. As such, blends of a high molecular weight PEG with a lower molecular weight PEG were attempted to allow a tailoring of properties. Formulations, with solids adjusted to give a maximum viscosity of ˜20 cP, prepared from straight 20KPEGA2, and blends of 20KPEGA2 with 10KPEGA2, and from a random copolymer of ethylene oxide and propylene oxide, 12KR, were sprayed onto a glass dish in-vitro, some on shaved rat skin content in-vivo, to yield films that were transparent, firm, adhered to skin, but were capable of being peeled off as an integral material even without the use of a drape. It is desirable that cure be rapid to prevent runoff and adherence due to mechanical interlocking of the curing gel in surface pores. Hydrogel formulations prepared from 20KPEGA2 and 35KPEGA2 with vinyl caprolactam (VC) comonomer and reduced hydrogen peroxide concentration were compared on glass.

The results appear at the end of Table 8. A blended formulation containing 5.4% 35KPEGA2 and 5.2% 20KPEGA2 was considered optimal in firmness and flexibility, imparting superior peelability.

In addition to the aforementioned properties, the PEG-based film material is biocompatible, non-toxic, non-irritating to the wound, permeable to gases, and can absorb large amounts of wound exudate, and can be delivered conformally by the spray technique

TABLE 8
Spray deposition, film characteristics and peelability of PEG-
based films on glass and/or rat skin.

| | [Ferrons | | | |
|---|---|---|---|---|
| | Gluconate] | [Ascorbic | [HOOH] | |
| Macromers | ppm | Acid] ppm | ppm | Film Quality |
| 20% 10K | 1000 | 3000 | 560 | Firm, peelable, |
| | | | | brittle |
| 15% 20K | 1000 | 3000 | 560 | Soft, peelable, |
| | | | | tougher |
| 10% 10K | 1000 | 3000 | 560 | Firm, peelable, |
| 7.5% 20K | | | | brittle |
| 6% 10K | 1000 | 3000 | 560 | Firm, peelable, |
| 10.5% 20K | | | | brittle |
| 4% 10K | 1000 | 3000 | 560 | Firm, peelable, |
| 12% 20K | | | | brittle |
| 2% 10K | 1000 | 3000 | 560 | Firm, peelable, |
| 13.5% 20K | | | | brittle |
| 1% 10K | 1000 | 3000 | 560 | Firm, peelable, |
| 14.3% 20K | | | | brittle |
| 0.5% 10K- | 1000 | 3000 | 560 | Firm, peelable, |
| 14.7% 20K | | | | tougher |
| 14.7% 20K | 1000 | 3000 | 560 | Peelable on rat skin |
| 14.7% 20K | 1000 | 3000 | 560 | Peelable on rat skin |
| 0.5% 10K | | | | |
| 8% 20K | 1000 | 3000 | 200 | Peelable; less firm |
| 0.9% NaCl | | | | |
| 12% 10K | 1000 | 3000 | 560 | Peelable, brittle |
| 0.9% NaCl | | | | |
| 10% 35K | 1000 | 3000 | 100 | Peels, very flexible |
| 0.4% VC | | | | but soft; slightly |
| | | | | weak |
| 14% 20K | 1000 | 3000 | 100 | Peels, but has a bit |
| 0.4% VC | | | | of brittle character |
| 5.4% 35K | 1000 | 3000 | 100 | Peels best; good |
| 5.2% 20K | | | | flexibility + strength |
| 0.4% VC | | | | |

Modifications and variations of the present invention will be apparent to those skilled in the art from the forgoing detailed description. All modifications and variations are intended to be encompassed by the following claims. All publications, patents, and patent applications cited herein are hereby incorporated by reference in their entirety.

What is claimed is:

1. A hydrogel wound dressing formed by spray delivery of a liquid composition to the wound, wherein the composition forms a hydrogel in situ on the wound.

2. The wound dressing of claim 1, wherein the hydrogel is degradable.

3. The wound dressing of claim 1, wherein the composition is delivered via an aerosol delivery device.

4. The wound dressing of claim 1, wherein the composition is delivered via a pump spray delivery device.

5. The wound dressing of claim 1, wherein the composition comprises water soluble macromers having one or more crosslinkable groups that crosslink in situ to form a hydrogel.

6. The wound dressing of claim 1, wherein the composition includes a macromer or polymer that gels in situ in response to a gelling stimulus.

7. The composition of claim 1, wherein the composition comprises a polymer that precipitates to form a hydrogel upon application to the wound.

8. The wound dressing of claim 1, wherein the composition further contains one or more additives selected from the group consisting of preservatives, biologically active agents, defoamers, wettings agents, leveling agents, hydrating agents, thickeners, fillers, and absorbents.

9. The wound dressing of claim 8, wherein the active agent is selected from the group consisting of growth factors (e.g. platelet-derived growth factor, epidermal growth factor, transforming growth factor beta (TGF-β)), nitric oxide, antibiotics, anti-inflammatories, analgesics, blood coagulants, and enzymes.

10. The wound dressing of claim 8, wherein the active agent is one which delivers NO to the wound.

11. The wound dressing of claim 1, wherein the dressing debrides the wound when it is removed.

12. The wound dressing of claim 6, wherein the water soluble macromers having one or more crosslinkable groups are PVA macromers modified with pendant crosslinkable groups.

13. The wound dressing of claim 6, wherein the in situ crosslinking is in response to redox initiation.

14. A method of forming a hydrogel wound dressing, comprising the steps:
applying a composition to a wound via spray; and
bringing a gelling stimulus into contact with the composition, either before, during, or after application of the composition to the wound, causing formation of the hydrogel wound dressing.

15. The method of claim 14, wherein the hydrogel is degradable.

16. The method of claim 14, wherein the composition is delivered via an aerosol delivery device.

17. The method of claim 14, wherein the composition is delivered via a pump spray delivery device.

18. The method of claim 14, wherein the composition comprises water soluble macromers having one or more crosslinkable groups that crosslink in situ to form a hydrogel.

19. The method of claim 14, wherein the composition includes a macromer or polymer that gels in situ in response to a gelling stimulus.

20. The method of claim 14, wherein the composition comprises a polymer that precipitates to form a hydrogel upon application to the wound.

21. The method of claim 14, wherein the composition further contains one or more additives selected from the group consisting of preservatives, biologically active agents, defoamers, wettings agents, leveling agents, hydrating agents, thickeners, fillers, and absorbents.

22. The method of claim 21, wherein the active agent is selected from the group consisting of growth factors (e.g. platelet-derived growth factor, epidermal growth factor, transforming growth factor beta (TGF-β)), nitric oxide, antibiotics, anti-inflammatories, analgesics, blood coagulants, and enzymes.

23. The method of claim 21, wherein the active agent is one which delivers NO.

24. The method of claim 18, wherein the water soluble macromers having one or more crosslinkable groups are PVA macromers modified with pendant crosslinkable groups.

25. The method of claim 14, wherein the in situ polymerization is in response to redox initiation.

26. A composition for forming a hydrogel wound dressing comprising a macromer or polymer that gels in situ to form a hydrogel.
